(19) United States
(12) Patent Application Publication
(10) Pub. No.: US 2023/0371855 A1
(43) Pub. Date: Nov. 23, 2023

(54) METHOD OF EVALUATING AGING LEVEL OF A HUMAN THROUGH MEASURING VASCULAR STRUGGLE IN AN AGING HUMAN TISSUE

(71) Applicant: NuLiv Science USA, Inc., Brea, CA (US)

(72) Inventors: Chia-Hua Kuo, Taipei (TW); Michael, Chang Yu Wang, Brea, CA (US)

(73) Assignee: NuLiv Science USA, Inc., Brea, CA (US)

(21) Appl. No.: 18/199,048

(22) Filed: May 18, 2023

Related U.S. Application Data
(60) Provisional application No. 63/321,249, filed on Mar. 18, 2022.

(51) Int. Cl. A61B 5/1455; A61B 5/00; A61B 5/22

(52) U.S. Cl. CPC A61B 5/14553; A61B 5/6814; A61B 5/225; A61B 5/7239

(57) ABSTRACT

The present invention provides a method of evaluating vascular struggle in an aging human tissue and aging level during muscle exertion. The method includes the steps of: placing an optical detector on the person's brain while the person sits in a chair and place a hand on a table; asking the person to relax until a stable baseline is established by the optical sensor; turning on a pacer during a 30-second rest phase before asking the person to perform a handgrip test; asking the person to grip at maximal effort every three seconds during a 30-second grip phase while a total of 10 grips are performed and corresponding brain oxygenation and hemoglobin fluctuations are captured; asking the person to relax for a recovery phase while brain oxygenation and hemoglobin fluctuations are continued being captured for 30 seconds; repeating the entire procedure with a three-minute interval; transforming captured data into variability to reflect the amplitude of oxygenation and hemodynamic fluctuation; and evaluating the aging level of the person with greater fluctuation in hemodynamic variability corresponds to greater aging.

### CROSS REFERENCE TO RELATED APPLICATIONS

This non-provisional application claims the benefit under 35 U.S.C. § 119(e) to U.S. Provisional Application No. 63/321,249, filed on Mar. 18, 2022, which is hereby expressly incorporated by reference into the present application.

### FILED OF THE INVENTION

The present invention concerns a method of evaluating aging level of a human through measuring vascular struggle in a tissue during muscle exertion,

### BACKGROUND

In human body, blood vessels transport oxygen around to different regions internally. However, vascular endothelial function is known to decline with age. For a human tissue to maintain its function, increased metabolic activity has been observed clinically. Such increased activity can be determined by the real-time changes of blood distribution in a tissue. To monitor this change, typically a non-invasive near-infrared spectroscopy (NIRS) using a single detector probe is adopted. During the NIRS measurement, blood distribution in the capillaries of a tissue can be optically detected by tracing mobile hemoglobin concentration (oxy- and deoxy-hemoglobin) at high frequency, whilst the oxygenation levels (% oxygen saturation) can be calculated as the oxyhemoglobin-to-total hemoglobin ratio during the same measuring period. A technical shortfall of the NIRS measurement is different baseline values in the tissue of interest associated with site-to-site variations in cytochrome levels, which limits inter-tissue and inter-individual comparison.

It is possible and desirable to develop a method and system to evaluate the aging level of a human through NIRS measurement.

### SUMMARY OF THE INVENTION

Accordingly, the invention provides a method and system for evaluating ageing level of a human, through the evaluation of vascular struggle in living human tissues.

In the invention, to address the problem of discrepancy between decreased vascular endothelial function and increased brain metabolic activity during aging [3], we examined the magnitudes of fluctuation in total hemoglobin (HV) and % oxygen saturation (OV) at rest and during a maximal voluntary handgrip test were examined for 834 adults across a wide age range (20 to 88 y). The maximal effort-handgrip test at as a stressor and measure the magnitude of vascular change at this condition. This is allowing us to quantitate the magnitude of vascular struggle to maintain oxygenation in a living tissue and ask the question whether this indicative alters with age.

In one aspect, the invention provides a method of evaluating aging level of a subject, which includes:
capturing brain oxygenation and hemoglobin fluctuations of the subject by an optical detector while the subject is relaxing until a stable baseline is established by the optical sensor, and then the subject is asked to grip at maximal effort every three seconds during a 30-second grip phase while a total of 10 grips are performed; and then to relax for a recovery phase while brain oxygenation; repeating the entire procedure with a three-minute interval;
transforming captured data into variability analysis to reflect the amplitude of oxygenation and hemodynamic fluctuation; and
evaluating the aging level of the subject by the fluctuation of hemodynamic variability as obtained, wherein the greater fluctuation of hemodynamic variability corresponds to the greater aging.

In one embodiment, the brain oxygenation and hemoglobin fluctuations are captured at a frequency more than 1 Hz.

According to the invention, the variability analysis can be performed by any applicable mathematic algorithms such as standard deviation.

### BRIEF DESCRIPTION OF THE DRAWINGS

FIG. 1 shows a method of evaluating vascular struggle in an aging human tissue and aging level during muscle exertion.

FIG. 2 shows cerebral hemodynamic response during a maximal handgrip strength test.

FIG. 3 shows the age breakdown of brain hemodynamic response against a maximal handgrip strength test.

FIG. 4 shows the scatter plots of oxygenation variability (OV) and hemodynamic variability (HV) during a maximal handgrip strength test of 834 adults aged 20-88 y.

FIG. 5 shows the maximal voluntary muscle strength and hemodynamic variability at higher ages.

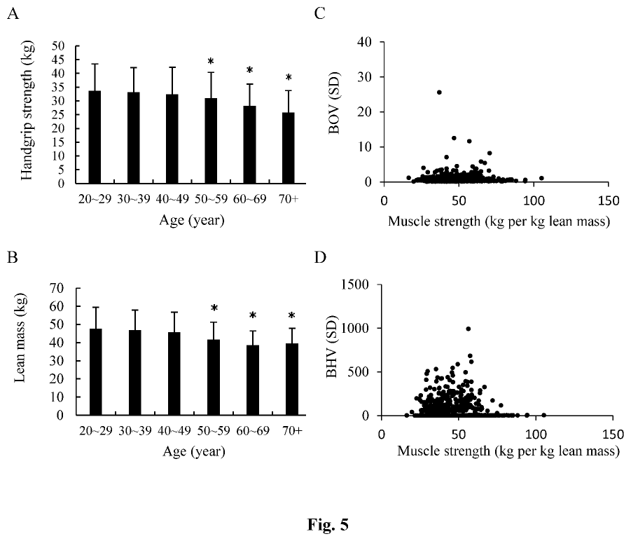

### DETAILED DESCRIPTION

Unless otherwise defined, all technical and scientific terms used herein have the same meaning as commonly understood by those skilled in the art to which the present invention pertains. It should be understood that the terminology used herein is for the purpose of describing specific embodiments only, and is not intended to be limiting.

It is to be understood that both the foregoing general description and the following detailed description are exemplary and explanatory only and are not restrictive of the invention.

The present invention relates to determining how tissue oxygenation stability is maintained during a maximal voluntary muscle contraction effort and whether the hemodynamic control to maintain this oxygenation stability is compromised at higher age.

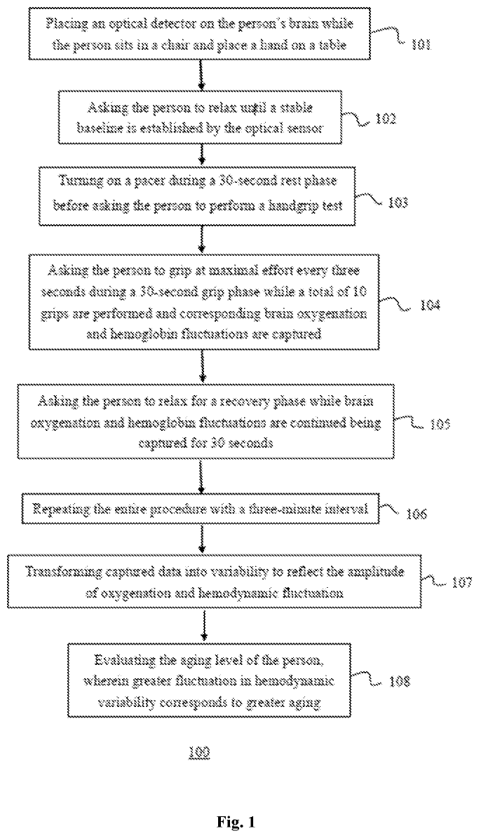

According to one embodiment, a method 100 of evaluating vascular struggle in an aging human tissue and a person's aging level during muscle exertion is provided. The method, as best shown in FIG. 1, in 101, placing an optical detector on the person's brain while the person sits in a chair and place a hand on a table, in 102 asking the person to relax until a stable baseline is established by the optical sensor, in 103, turning on a pacer during a 30-second rest phase before asking the person to perform a handgrip test, in 104, asking the person to grip at maximal effort every three seconds during a 30-second grip phase while a total of 10 grips are performed and corresponding brain oxygenation and hemoglobin fluctuations are captured, in 105, asking the person to relax for a recovery phase while brain oxygenation and hemoglobin fluctuations are continued being captured for 30 seconds, in 106, repeating the entire procedure with a three-minute interval, in 107, transforming captured data into variability to reflect the amplitude of oxygenation and hemodynamic fluctuation, and, in 108, evaluating the aging level of the person, where greater fluctuation of hemodynamic variability corresponds to greater aging.

Prior to evaluation, a total of 834 participants (height 164.9±9.2 cm, weight 65.0±14.1 kg) from Taipei City aged 20-88 y were asked to report to a laboratory on a single occasion. Exclusion criteria were inability to conduct a maximal handgrip strength test and orthopedic implantation of metal. They were required to abstain from strenuous exercise and alcohol consumption 48 h prior to the experimental session. For the entire duration of the experimental procedures, participants were required to remove any accessories. They were familiarized with all the experimental procedures protocol and researcher corrected the procedure, if needed. They were asked to identify their dominant arm.

Participants gave their written informed consent before the test and University of Taipei Institutional Review Board approved the study protocol (IRB-2018-073). All experimental procedures were conducted in accordance with the spirit of Declaration of Helsinki.

To standardize the protocol in preventing difference in maximal muscle strength due to the angle of the elbow, participants sat in a chair at the most comfortable upright position and put hand straight on a table in the front during the entire testing procedure with a few adjustments. Maximal handgrip muscle strength was measured using a Takei Dynamometer Model T.K.K.5401 (Takei Scientific Instruments Co., Niigata, Japan). Participants practiced using the dynamometer and were then informed about the 3 phases of the measurements: rest (30 sec), 10-grips (30 sec), recovery (30 sec). Cerebral oxygenation variability (OV) and hemodynamic variability (HV) can be measured by a non-invasive near-infrared spectroscopy (NIRS) wireless device (PortaLite, Artinis Medical System, Elst, Netherlands) before, during, and after the handgrip muscle strength test. A NIRS optical detector is placed on the left frontal region if dominant arm was right hand, vice versa.

Since mental and physical disturbance can vary NIRS values in the brain, experimenters paid special attention to calm participants before establishing a baseline. Participants were asked to relax completely until a stable baseline was established for 1 min before recording the 30-s rest phase. The baseline is defined by minimal fluctuations in oxygenation (% oxygen saturation) and total hemoglobin without an obvious upward or downward trend for 1 min. During the 30-s rest phase, pacer was turned on 10 sec before the first grip. During contraction phase, participants started the grip at maximal effort every 3 sec (fingers always attached the device) according to the pacer. Following the handgrip test, brain hemodynamic changes continued to record for another 30 sec during the recovery phase. The entire procedure was repeated with a 3 min rest interval for the purpose to determine the test-retest reliability. The test-retest reliability in HV was 0.71 (p<0.01).

Data of total hemoglobin and oxygenation (% oxygen saturation) were collected at >1 Hz via bluetooth using Oxysoft software (Artinis Medical Systems, Elst, Netherlands). To reflect the amplitude of hemodynamic fluctuation, a total 300 values (1 Hz in 30 sec) of real-time data were transformed into variability (standard deviation, SD) of total hemoglobin (HV) and oxygenation (OV) for each participant. The transformation of optical data into variability is not limited to standard deviation technique, any applicable mathematical algorithms such as variance, range, interquartile range are included. Such variables allow quantifying the magnitude of hemodynamic oscillation to maintain oxygenation stability. In this study, the novel dimensionless indicatives, namely oxygenation variability (OV) and hemodynamic variability (HV), were developed by transforming a series of real-time data into standard deviation values to reveal the effort of vascular system for maintaining oxygen homeostasis in the human tissue. The algorithm takes the square root of the mean of the squared deviations of the optical values subtracted from their mean value. Therefore, both indicators allow quantitating fluctuation of a set of mobile hemoglobin and oxygenation values for different participants regardless their baseline level. Low OV represents a sufficient control of the vascular system towards a set point of oxygen homeostasis in the tissue. HV reflects the magnitude of vascular oscillation to maintain stable oxygenation (at a low OV). Since mobile hemoglobin values are a direct estimate of blood concentration in tissues, HV reflects the magnitude of vascular regulation resulted from fast vasoconstriction and vasorelaxation to maintain tissue oxygenation (i.e. low OV reflects high oxygen stability).

Shapiro-Wilk test was used to test whether all variables were normally distributed. Homogeneity of variances was confirmed by Levene test. Since the data for absolute total hemoglobin and tissue oxygenation levels (oxyhemoglobin-to-total hemoglobin ratio) are not normally distributed, OV and HV are expressed as median (95% confidence interval or CI). To indicate the percentage of the population exceeding the upper ceiling of normal range for HV for each age levels, we considered extreme values as 95% CI above the young age group (20-29 y) and was tested by Chi square. Friedman test was used and when a significant F ratio was found, Mann-Whitney U test was used for post hoc analysis. A level of significance was set at an α=0.05 for all variables, and values are expressed as median (95% CI). Mean value of 10 maximal handgrip strength was used for statistical analysis. Pearson correlation was calculated for the variables between the first handgrip test and the second handgrip test. SPSS 25.0 was used for statistical analysis.

Sex stratification and anthropometrical characteristics of the participants are shown in Table 1.

TABLE 1
Participant characteristics.

| N = 834 | Age 20-50 | | Age 51-88 | | p |
|---|---|---|---|---|---|
| Sex | M/F: 245/235 | | M/F: 141/213 | | — |
| Weight (kg) | 67 | (38-132) | 62 | (39-113) | <0.001 |
| Height (cm) | 168 | (147-195) | 161 | (143-187) | <0.001 |
| BMI | 23.8 | (16.0-48.9) | 23.8 | (15.0-37.6) | NS |
| Lean mass (kg) | 46.7 | (24.6-83.4) | 39.8 | (24.9-72.5) | <0.001 |
| Fat mass (kg) | 17.7 | (4.7-64.6) | 19.3 | (6.0-41.4) | 0.001 |
| Bone mass (g) | 2.61 | (1.38-4.21) | 2.14 | (1.18-3.89) | <0.001 |
| BMD (g/cm²) | 1.23 | (0.80-1.61) | 1.08 | (0.69-1.54) | <0.001 |
| OV-Q1 | 0.26 | (0.10-0.33) | 0.23 | (0.10-0.34) | NS |
| OV-Q2 | 0.42 | (0.33-0.51) | 0.42 | (0.34-0.51) | NS |
| OV-Q3 | 0.66 | (0.51-0.86) | 0.59 | (0.51-0.78) | NS |
| OV-Q4 | 1.30 | (0.86-11.64) | 1.02 | (0.79-3.26) | NS |
| HV-Q1 | 0.30 | (0.01-0.42) | 0.76 | (0.01-42) | <0.001 |
| HV-Q2 | 0.56 | (0.43-0.72) | 66 | (43-96) | <0.001 |
| HV-Q3 | 0.90 | (0.73-1.24) | 120 | (96-173) | <0.001 |
| HV-Q4 | 1.91 | (1.25-186) | 260 | (174-683) | <0.001 |

Data is expressed as median (95% confidence interval). Abbreviation: BMI, body mass index; BMD, bone mineral density; OV, oxygenation variability at rest; HV, hemodynamic variability at rest. Lower resting values of OV and HV represent higher hemodynamic stability. Q1-Q4 represents each quartile of the age range investigated.

Resting OV values were consistently low and similar for participants across the entire age range (age 20-88 y). While no difference in median OV was found before and after age 50 y, median HV was substantially greater in participants aged >50 y than those younger age counterparts for each quartile. FIG. 1 shows examples of cerebral hemodynamic response during maximal handgrip muscle contractions (10 repetitions in 30 sec) of men aged 25 y and 75 y, respectively. The amplitude of fluctuations in cerebral oxygenation (oxyhemoglobin-to-total hemoglobin ratio, OV) against the 30-sec muscle contraction was small (A, B), whereas cerebral hemoglobin fluctuation was increased substantially above the rest phase (C, D) for both age levels. While the response patterns against muscle exertion are similar for both ages, the scale of such changes in cerebral hemoglobin during a maximal handgrip muscle strength test was substantially greater for the adult aged 75 y (D) than 25 y (C), indicating a greater vascular struggle in the older adult to maintain a stable oxygenation in the brain.

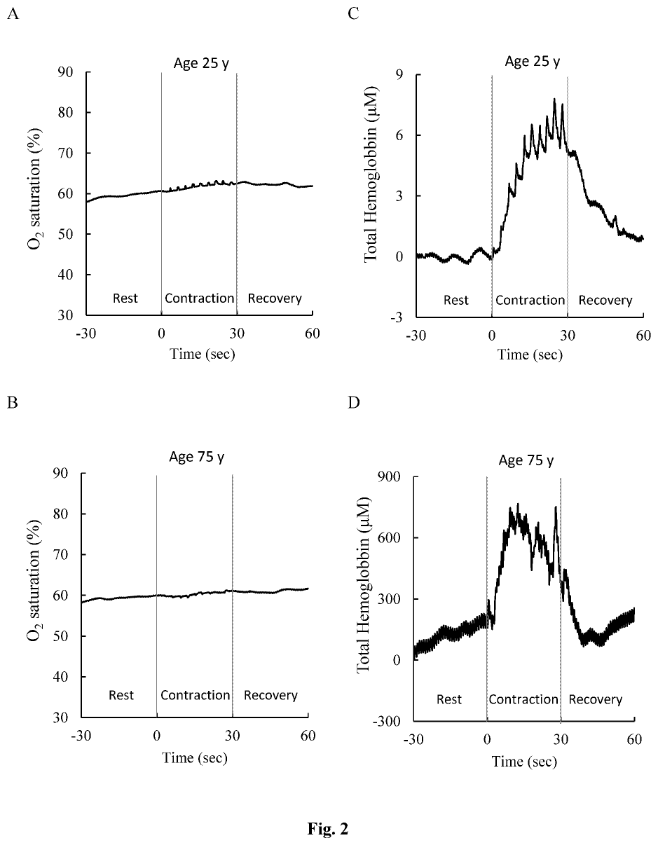

Age breakdown of OV and HV responses against maximal handgrip muscle contractions are presented separately in FIG. 2. A significant main effect of exercise response for both OV and HV was consistently observed (rest vs. contraction, α<0.01). This acute response of OV and HV returned quickly during the 30-s recovery period (contraction vs. recovery, α<0.05). Small increases in OV above resting levels (+21% and +33%) during a maximal handgrip muscle strength test were observed in the young adults aged <40 y (A, B) (main effect of time, α<0.05). From age 40-88 y, the muscle contraction-induced increases in OV above rest were doubled (C, D, E, F) (main effect of time, α<0.05). The muscle contraction-induced increases in HV were mostly >100% above the rest phase at all age levels (G, H, I, J, K, L). Despite a similar pattern across all ages, the scale of the HV changes for adults aged after 50 y was >100 folds of their young age counterparts (main effect of time, α<0.001).

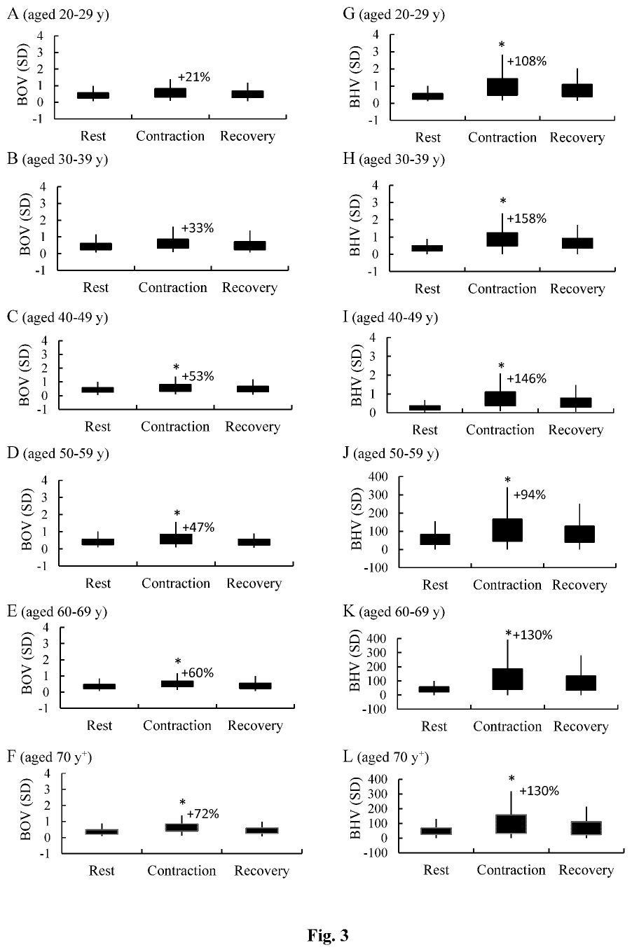

FIG. 3 shows scatter plots of OV and HV values of all participants (n=834) from age 20 to 88 y. This result indicates an apparently low OV values during rest (A), contraction (B), and recovery (C) (stable oxygenation levels) compared with HV for participants aged 20-88 y, suggesting a preservation of high stability in brain oxygenation across a wide age range. HV values in the individuals aged above 50 y (D, E, F) were much higher than their young age counterparts (aged 20-50 y). A sharp cutoff point after age 50 for the age-dependent increases in HV was very prominent for both men and women (G, H). Therefore, potential bias is unlikely. The ratio of population escaped from normal range (95% CI of 20-29 y) of HV values does not appear to increase until age 50 y. In particular, the percentage of the population exceeding the upper ceiling of normal range (95% CI) of their young age counterparts are 11% (20-29 y), 11% (30-39 y), and 7% (40-49 y), 85% (50-59 y), 81% (60-69 y), and 80% (70+y) (α<0.001). The sharp cut-off point of HV after age 50 y is also similar at rest and recovery phases (D, F).

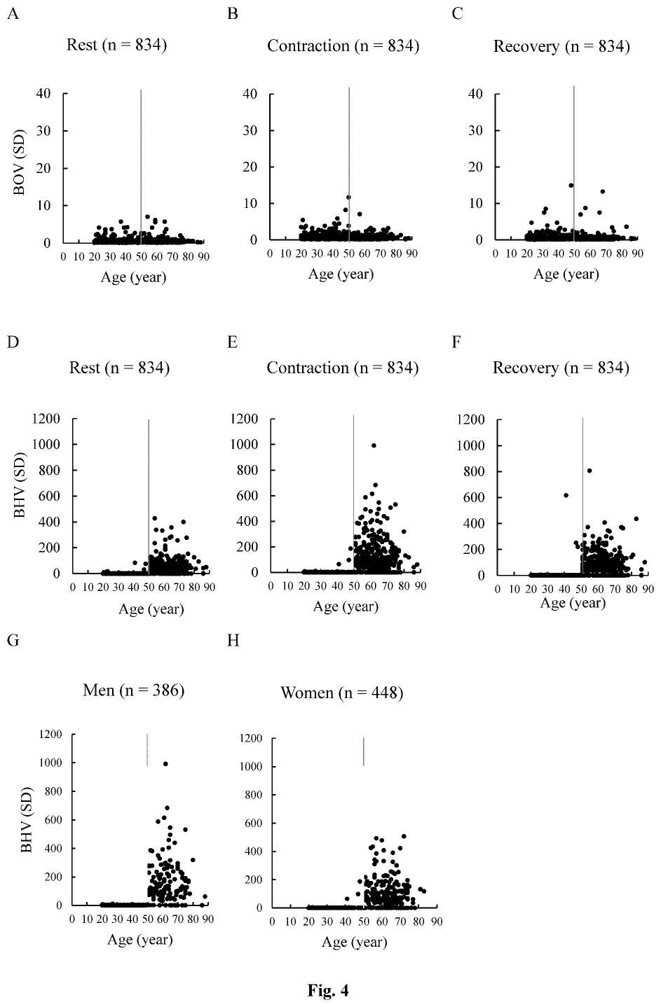

FIG. 4 presents the average values of maximal handgrip muscle strength and lean body mass of participants from age 20 to 88 y. Adults aged >50 y had slightly lower maximal handgrip muscle strength (A) and lean body mass (B) compared with those at younger aged 20-29 y (α<0.05). The scatter plot shows a non-linear relationship between OV and muscle strength (kg per kg body weight in percentage) (C). Adults with the highest muscle strength (>75 kg per kg body weight) showed very low incidence of both high OV and high HV compared with those with low muscle strength counterparts (D).

Vascular endothelial function is known to decrease with age. Intriguingly, metabolic activity of the prefrontal brain elevates with age. This discrepancy implicates an age-dependent compensatory effort to sustain oxygenation in the brain. Oxygen delivery to match metabolic demand among cells relies on fast vasorelaxation and vasoconstriction by flashing oxygenated blood in tissues. To address the discrepancy, we developed OV and HV to indicate the magnitude of real-time fluctuation of oxygenation and blood distribution in the prefrontal brain. The experimental procedures validated that HV and OV as indicators for brain vascular struggle using maximal voluntary handgrip exertions. The major findings of the study are as follows: 1) HV increased substantially while OV increase to less extent during an episode of muscle contractions at a maximal effort; 2) HV at rest and during maximal handgrip contractions increased >100 folds after age 50 y, suggesting an amplified vasorelaxation and vasoconstriction effort to maintain normal brain oxygenation stability (low OV); 3) Brain oxygenation stability during maximal handgrip contractions seems to be well-preserved and remained similar from age 20 to 88 y. These results provide a mechanistic explanation of increased metabolic activity of the prefrontal brain [16] as a physiological compensation during vascular aging, and indicates that the vascular aging accelerates after age 50 y. A sharp increase in HV indicates a huge hemodynamic compensation against a cliff-like vascular deterioration in the aging tissue, in contrast with our conventional view that aging is a gradually occurring process.

Maximal voluntary handgrip strength is a well-established indicative of brain health. In this study, further evidence was provided that indicates vascular control in the brain is directly involved with mobilization of muscles by central commands. In the study, we selected 10 attempts at maximal exertion efforts based on our preliminary test of brain hemoglobin elevation in which plateau cannot be achieved by 3 attempts for most of participants. Most of previous studies used 3 attempts to indicate the maximal voluntary effort during handgrip contractions. A very high correlation between average maximal muscle strength and the peak muscle strength of 10 attempts in our study suggests no difference of using both methods to obtain maximal handgrip strength.

In the present study, sharply increased oscillations in cerebral hemoglobin (reflected by increased HV) after age 50 y may be to compensate the loss of anaerobic capacity and/or deteriorated endothelial function. It is worthy to note that the age-dependent increases in HV after age 50 y is also evident at rest. A recent study from multi-tracer PET brain imaging suggests a metabolic shift from mixture of nonoxidative and oxidative use of glucose towards predominantly oxidative metabolism during advancing age [19]. Glycolysis decreases to nearly zero at the age of 60 y [19], suggesting that amplifying oscillation of blood distribution in the brain may have been a compensation to the loss of anaerobic metabolism to maintain ATP production. These results partly explain an increased oxygen extraction in the aging tissue [20]. Age-dependent deterioration in endothelial function is known as a major contributor for functional decline in older adults [13]. Endothelial cell senescence is expected to compromise efficiency of oxygen and nutrient transport to the brain. To adapt with this age-dependent deterioration, a hemodynamic compensation would be required to sustain cell survival in the brain.

A sharp increase in HV after age 50 implicit a fast deterioration occurring in the vascular system within a year period. The underlying mechanism accounted for the fast deterioration is far from clear. We speculate that this rapid decline is associated with an emergence of daily deficit in endothelial cell renewal. Endothelial cells in capillary of animal tissues have a short lifespan around 2 week. Therefore, a 100% daily endothelial cell replacement is required to maintain youth and functional vascular endothelium for oxygen delivery to surrounding neurons. Assuming a daily renewal rate of endothelial cells decreases from 100% to 98%, unreplaced senescent endothelial cells can rapidly accumulate to 87% in the entire population of capillary in 100 days, leading to a cliff-like vascular deterioration. The observed increases in HV with a relatively stable OV suggest an enormous vascular effort to shuffle more blood in and out of the brain tissue for oxygenation. Endothelial progenitor cells are produced from bone marrow to replace senescent endothelial cells in blood vessels. Previous findings show there is an age-dependent decrease in bone to body mass ratio during natural growth in middle-age, which may explain the emerged deficit in endothelial cell replacement due to an imbalanced development between the bone marrow cell production organ and an expanding body mass. Furthermore, loss of sex hormones at age 50 y in men and women may partly contribute to the corresponding vascular deterioration reflected by increased HV. Anabolic hormones like sex hormone and insulin play a key role in basal bone marrow cell proliferation to sustain vascular function.

The present study did not find age-dependent changes in HV across the age from 20 to 50 y. Following a sharp increase in HV after age 50, no further age-dependent changes in HV was observed thereafter. The possibility that there may be a ceiling for survival in the vascular compensation by amplified HV after age 50 y can not be precluded. This means that when HV is elevated to an extent of compensation failure for oxygen homeostasis, the individuals can no longer survive, and absent as a participant in the study. A longitudinal observation for a wide population around age 50 y would be needed to clarify this possibility.

The present study validates OV and HV as novel indicatives to reflect the ability to maintain oxygenation and blood distribution, respectively, in the brain. Low levels of OV and HV represent better oxygenation stability with a relaxed effort of vascular controls. Maximal voluntary muscle contraction increased HV, and to a minor extent, OV suggesting a priority to sustain brain oxygenation within a narrow range in cost of increasing hemodynamic control. The most striking finding of the study is a sharp increase in HV after age 50 y for men and women at rest and during muscle contraction, while OV remains quite stable across a wide age range. This huge compensation effort after age 50 y implicates the emergence of age-onset deficit in daily replacement of short-lived endothelial cells leading to accumulations of senescent/unfunctional endothelial cell population in the vasculatures within a year.

What is claimed is:

1. A method of evaluating vascular struggle in an aging human tissue and a person's aging level during muscle exertion, comprising:
placing an optical detector on the person's brain while the person sits in a chair and place a hand on a table;
asking the person to relax until a stable baseline is established by the optical sensor;
turning on a pacer during a 30-second rest phase before asking the person to perform a handgrip test;
asking the person to grip at maximal effort every three seconds during a 30-second grip phase while a total of 10 grips are performed and corresponding brain oxygenation and hemoglobin fluctuations are captured;
asking the person to relax for a recovery phase while brain oxygenation and hemoglobin fluctuations are continued being captured for 30 seconds;
repeating the entire procedure with a three-minute interval;
transforming captured data into variability to reflect the amplitude of oxygenation and hemodynamic fluctuation; and
evaluating the aging level of the person, wherein greater fluctuation in hemodynamic variability corresponds to greater aging.

2. The method of claim 1, wherein the brain oxygenation and hemoglobin fluctuations are captured at a frequency of >1 Hz.

3. The method of claim 1, wherein variability analysis is performed by standard deviation and Shapiro-Wilk test is used to test whether all variables are normally distributed.
